(19) United States
(12) Patent Application Publication
(10) Pub. No.: US 2015/0337039 A1
(43) Pub. Date: Nov. 26, 2015

(54) MAC-1 ANTIBODIES AND USES THEREOF

(71) Applicant: Case Western Reserve University, Cleveland, OH (US)

(72) Inventor: Daniel I. Simon, Cleveland, OH (US)

(21) Appl. No.: 14/719,984

(22) Filed: May 22, 2015

Related U.S. Application Data
(60) Provisional application No. 62/001,973, filed on May 22, 2014.

(51) Int. Cl. C07K 16/28

(52) U.S. Cl. CPC C07K 16/2839; C07K 2317/34; C07K 2317/92; C07K 2317/14; C07K 2317/565; C07K 2317/76; A61K 2039/505

(57) ABSTRACT

A method of treating a subject having or at risk of a disease or disorder associated with aberrant leukocyte-platelet interactions includes administering to the subject a therapeutically effective amount of a monoclonal antibody or antigen binding portion thereof that specifically binds to at least one of SEQ ID NO: 1, SEQ ID NO: 2, or SEQ ID NO: 3 to inhibit leukocyte arrest on adherent platelets in the subject.

### RELATED APPLICATION

This application claims priority from U.S. Provisional Application No. 62/001,973, filed May 22, 2014, the subject matter of which is incorporated herein by reference in its entirety.

### FIELD OF THE INVENTION

The present invention relates to antibodies specific to Mac-1 and to the use of such antibodies to treat disease and/or disorders associated with aberrant leukocyte-platelet interactions.

### BACKGROUND

Leukocyte-platelet interactions are critical in the initiation and progression of atherosclerosis as well as restenosis. Platelet deposition precedes inflammatory cell accumulation in mouse models of atherogenesis, and inhibition of platelet adhesion dramatically reduces atherosclerotic lesion formation. However, the specific receptors responsible for mediating adhesive interactions between neutrophils and platelets in vivo are incompletely defined.

Recruitment of circulating leukocytes to vascular endothelium requires multistep adhesive and signaling events including selectin-mediated attachment and rolling, leukocyte activation, and integrin-mediated firm adhesion and diapedesis that result in the infiltration of inflammatory cells into the blood vessel wall. Firm attachment is mediated by members of the β₂-integrin family, LFA-1 (α_{L}β₂, CD11a/CD18), Mac-1 (α_{M}β₂, CD11b/CD18), and p150,95 (α_{X}β₂, CD11c/CD18), which bind to endothelial counterligands (e.g., intercellular adhesion molecule-1 [ICAM-1]), to endothelial-associated extracellular matrix proteins (e.g., fibrinogen), or to glycosaminoglycans.

### SUMMARY

Embodiments described herein relate to monoclonal antibodies and antigen binding fragments thereof and their use in methods of treating diseases or disorders associated with aberrant leukocyte-platelet interactions. It was found that the interaction of leukocyte Mac-1 with platelet GP Ibα is broadly required for inflammation and initiates pro-inflammatory and pro-thrombotic signals that promote diverse disease processes.

Administration of monoclonal antibodies or antigen binding fragments thereof, which inhibit interaction of leukocyte Mac-1 with platelet GP Ibα, to subjects having or at risk of disease or disorders associated with aberrant leukocyte-platelet interactions can be used treat the disease or disorder.

In some embodiments, the diseases or disorders associated with aberrant leukocyte-platelet interactions can include post-cardiopulmonary bypass inflammation, pathogenic hypertrophy, cardiopulmonary bypass, percutaneous coronary intervention (PTCA), ischemia-reperfusion following acute myocardial infarction, myocardial infarction, atherosclerosis, heparin-induced extracorporeal membrane oxygenation LDL precipitation, extracorporeal membrane oxygenation, multiple organ failure, thrombosis formation, neointimal formation, neointimal hyperplasia, atherothrombosis, vasculitis, restinosis, stroke, angina, arthritis, and demyelinating disorders, such as multiple sclerosis and amyotrophic lateral sclerosis.

In other embodiments, the monoclonal antibody or antigen binding fragment thereof specifically binds to at least one of SEQ ID NO: 1, SEQ ID NO: 2, or SEQ ID NO: 3. The antibody can have a nanomolar binding affinity (K_{D}) to Mac-1 of less than about 10 nanomoles, less than about 5 nanomoles, or less than about 1 nanomoles and a nanomolar inhibitory constant (Ki) of leukocyte Mac-1 binding to platelet GP Ibα less than about 10 nanomoles, less than about 5 nanomoles, or less than about 1 nanomoles.

The monoclonal antibody can be chimeric, recombinant, humanized, and/or de-immunized. The antibody can also be an antibody fragment, such as a single chain antibody, IgG, F(ab)2, F(ab′)2, F(ab), F(ab′) fragment, or truncated antibody (e.g., Fc truncated antibody).

In some embodiments, the monoclonal antibody or antigen binding fragment can be a humanized monoclonal antibody or antigen binding fragment thereof that includes the 3 CDRs of the heavy chain variable domain and the 3 CDRs of light chain variable domain of a rat monoclonal antibody that specifically binds to SEQ ID NO: 1, SEQ ID NO: 2, or SEQ ID NO: 3. The rat monoclonal antibody can be produced by clones 5C8, 10E2, 4F9, or 4B10.

The antibody can be produced in a cell, mammal, genetically engineered mammal, or by phage display.

Other embodiments, relate to a method of treating a subject by passing circulating blood from a blood vessel of the subject, through a conduit, and back to a blood vessel of the subject. The conduit has a luminal surface that includes a material capable of causing at least one of complement activation, platelet activation, leukocyte activation, or platelet-leukocyte adhesion in the subject's blood. A monoclonal antibody is introduced into the subject's bloodstream in an amount effective to reduce platelet-leukocyte adhesion resulting from passage of the circulating blood through the conduit. The passing of blood through the conduit can occur before and/or during and/or after introducing the monoclonal antibody into the subject's bloodstream.

In an aspect of the invention, the medical procedure can be an extracorporeal circulation procedure, such as a cardiopulmonary bypass procedure.

The antibody can be a monoclonal antibody or antigen binding fragment thereof that specifically binds to at least one of SEQ ID NO: 1, SEQ ID NO: 2, or SEQ ID NO: 3. The antibody can have a nanomolar binding affinity (K_{D}) to Mac-1 of less than about 10 nanomoles, less than about 5 nanomoles, or less than about 1 nanomoles and a nanomolar inhibitory constant (Ki) of leukocyte Mac-1 binding to platelet GP Ibα less than about 10 nanomoles, less than about 5 nanomoles, or less than about 1 nanomoles.

The monoclonal antibody can be chimeric, recombinant, humanized, and/or de-immunized. The antibody can also be an antibody fragment, such as a single chain antibody, IgG, F(ab)2, F(ab′)2, F(ab), F(ab′) fragment, or truncated antibody (e.g., Fc truncated antibody).

In some embodiments, the monoclonal antibody or antigen binding fragment can be a humanized monoclonal antibody or antigen binding fragment thereof that includes the 3 CDRs of the heavy chain variable domain and the 3 CDRs of light chain variable domain of a rat monoclonal antibody that specifically binds to SEQ ID NO: 1, SEQ ID NO: 2, or SEQ ID NO: 3. The rat monoclonal antibody can be produced by clones 5C8, 10E2, 4F9, or 4B10.

### BRIEF DESCRIPTION OF THE DRAWINGS

FIG. 1 illustrates alignment of human α_{M}(P²⁰¹-K²¹⁷) sequences (SEQ ID NO: 1) with corresponding mouse and rat sequences (SEQ ID NO: 2 and SEQ ID NO: 3).

FIGS. 2(A-C) illustrate targeting Mac-1-GP-1bα prevents myocardial infarction and prolongs survival.

FIG. 3 illustrates a chart showing deficiency of Mac-1 or disruption of Mac-1-GP-1bα prolongs carotid artery occlusion time after photochemical (Rose Bengal-green light laser) injury.

### DETAILED DESCRIPTION

As used herein, the term “acceptor human framework” refers to a framework comprising the amino acid sequence of a VL or VH framework derived from a human immunoglobulin framework, or from a human consensus framework.

As used herein, the term “antibody” covers full length monoclonal antibodies, polyclonal antibodies, nanobodies and multi-specific antibodies. Biological antibodies are usually hetero-tetrameric glycoproteins of about 150,000 Daltons, composed of two identical light (L) chains and two identical heavy (H) chains. The two heavy chains are linked together by disulfide bonds, and each heavy chain is linked to a light chain by a disulfide bond. Each full-length IgG molecule contains at least two binding sites for a specific target or antigen. Light chains are either kappa or the lambda. Both light chains contain a domain of variable amino acid sequences, called the variable region (variously referred to as a “V_{L},” “Vₖₐₚₚₐ,” or “V_{lambda}-region”) and a domain of relatively conserved amino acid sequences, called the constant region (“CL-region”). Similarly, each heavy chain contains a variable region (“V_{H}-region”) and three constant domains (“C_{H1}-,” “C_{H2}-,” and “C_{H3}-regions”) and a hinge region.

As used herein, the term “antibody fragment” refers to a segment of a full-length antibody, generally called as the target binding or variable region. Examples include Fab, Fab′, F(ab′)2 and Fv fragments. An “Fv” fragment is the minimum antibody fragment which contains a complete target recognition and binding site.

As used herein, the term “antigen binding fragment” refers to a fragment or fragments of an antibody molecule that contain the antibody variable regions responsible for antigen binding. Fab, Fab′, and F(ab)₂ lack the F_{C} regions. Antigen-binding fragments can be prepared from full-length antibody by protease digestion. Antigen-binding fragments may be produced using standard recombinant DNA methodology by those skilled in the art.

As used herein, complementarity-determining region (“CDR”) refers to a specific region within variable regions of the heavy and the light chain. Generally, the variable region consists of four framework regions (FR1, FR2, FR3, FR4) and three CDRs arranged in the following manner: NH₂-FR1-CDR1-FR2-CDR2-FR3-CDR3-FR4-COOH. The term “framework regions” refers to those variable domain residues other than the CDR residues herein defined.

The term “epitope” includes any protein determinant capable of specific binding to an immunoglobulin. Epitope determinants usually consist of chemically active surface groupings of molecules such as amino acids or sugar side chains and usually have specific three-dimensional structural characteristics, as well as specific charge characteristics.

The term “polypeptide” is used herein as a generic term to refer to native protein, fragments, or analogs of a polypeptide sequence. Hence, native protein, fragments, and analogs are species of the polypeptide genus.

The term “polypeptide fragment” as used herein refers to a polypeptide that has an amino-terminal and/or carboxyl-terminal deletion, but where the remaining amino acid sequence is identical to the corresponding positions in the naturally occurring sequence deduced, for example, from a full-length Mac-1 sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

As used herein, “Fab fragment” refers to the constant domain of the light chain and the first constant domain of the heavy chain. Fab′ fragments differ from Fab fragments by the few extra residues at the carboxyl terminus of the heavy chain CH1 domain including one or more cysteines from the antibody hinge region. F(ab′) fragments are produced by cleavage of the disulfide bond at the hinge cysteines of the F(ab′)₂pepsin digestion product.

As used herein, the term “functional fragment” of an antibody refers to an antibody fragment having qualitative biological activity in common with a full-length antibody. For example, a functional antibody fragment is one which can bind to properdin in such a manner so as to prevent or substantially reduce the alternative complement activation.

As used herein, the term “human consensus framework” refers to a framework which represents the most commonly occurring amino acid residue in a selection of human immunoglobulin VL or VH framework sequences. Generally, the selection of human immunoglobulin VL or VH sequences is from a subgroup of variable domain sequences.

As used herein, a “humanized antibody” refers to an antibody consisting of mostly human sequences, except for CDR1, CDR2, and CDR3. All framework regions are also humanized. A chimeric antibody comprises murine CDRs, murine framework regions, and human constant regions. Collectively, chimeric antibodies contain murine both variable regions and human constant regions.

As used herein, the term “identical” or “substantially identical” with respect to an antibody chain polypeptide sequence may be construed as an antibody chain exhibiting at least 65%, 70%, 80%, 90% or 95% sequence identity to the reference polypeptide sequence present in the variable region of the antigen binding fragment. The term with respect to a nucleic acid sequence may be construed as a sequence of nucleotides exhibiting at least about 65%, 75%, 85%, 90%, 95% or 97% sequence identity to the reference nucleic acid sequence.

As used herein, the term “individual” refers to a vertebrate, preferably a mammal and more preferably a human.

As used herein, the term “mammal” refers to any animal classified as a mammal includes humans, higher primates, domestic and farm animals, horses, pigs, cattle, dogs, cats and ferrets, etc. In one embodiment of the invention, the mammal is a human.

The term “monoclonal” refers to an antibody that binds to a sequence of amino acid and has a single specific epitope on its target antigen. For example, ant:-rM2 is a monoclonal antibody that is specific only to the P²⁰¹-K²¹⁷ amino acid sequence of Mac-1. Because the antibody is monoclonal, it would recognize a domain/motif that contains the sequence contained in 201-217 peptide (SEQ ID NO: 1).

The term “polyclonal” refers to an antibody that recognizes multiple epitope sites on a single antigen. For example, a polyclonal antibody against Mac-1 indicates that the antibody will bind several sites of the Mac-1 protein.

The terms “patient,” “mammalian host,” “subject”, and the like are used interchangeably herein, and refer to mammals, including human and veterinary subjects.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

It is to be understood that this invention is not limited to particular embodiments described, as such may, of course, vary. It is also to be understood that the terminology used herein is for describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are encompassed within the invention, subject to any specifically excluded limit in the stated range. Where the stated range includes one or both of the limits, ranges excluding either or both of those included limits are also included in the invention.

Embodiments described herein relate to monoclonal antibodies and antigen binding fragments thereof and their use in methods of treating diseases or disorders associated with aberrant leukocyte-platelet interactions. It was found that the interaction of leukocyte Mac-1 with platelet GP Ibα is broadly required for inflammation and initiates pro-inflammatory and pro-thrombotic signals that promote diverse disease processes. Experimental and clinical studies support an important linkage between inflammation and thrombosis. Monocytes are the major source of blood tissue factor (TF), and leukocyte-platelet interactions induce bi-directional signals that amplify pro-inflammatory and pro-thrombotic cellular responses.

Administration of monoclonal antibodies or antigen binding fragments thereof, which inhibit interaction of leukocyte Mac-1 with platelet GP Ibα, to subjects having or at risk of disease or disorders associated with aberrant leukocyte-platelet interactions can be used to treat the diseases or disorders.

In some embodiments, the diseases or disorders associated with aberrant leukocyte-platelet interactions can include post-cardiopulmonary bypass inflammation, pathogenic hypertrophy, cardiopulmonary bypass, percutaneous coronary intervention (PTCA), ischemia-reperfusion following acute myocardial infarction, myocardial infarction, atherosclerosis, heparin-induced extracorporeal membrane oxygenation LDL precipitation, extracorporeal membrane oxygenation, multiple organ failure, thrombosis formation, neointimal formation, neointimal hyperplasia, atherothrombosis, vasculitis, restinosis, stroke, angina, arthritis, and demyelinating disorders, such as multiple sclerosis and amyotrophic lateral sclerosis.

In some embodiments, a monoclonal antibody or antigen binding fragment thereof, which inhibits interaction of leukocyte Mac-1 with platelet GP Ibα, can specifically bind to the M2 region of α_{M}β₂ (Mac-1). The amino acid sequence of the human M2 region (α_{M}(P²⁰¹-K²¹⁷) (SEQ ID NO: 1)) is substantially homologous with the amino acid sequence of corresponding mouse and rat M2 sequences (SEQ ID NO: 2 and SEQ ID NO: 3). Antibodies can be generated that specifically bind to mouse or rat M2 (SEQ ID NO: 2 or SEQ ID NO: 3) and cross-react with or bind to human M2 (SEQ ID NO: 1) with similar or higher binding affinity. Monoclonal antibodies or antigen binding fragments thereof can then be screened for their ability to bind to rat macrophage cell line (NR8383), mouse macrophage cell line (RAW) and 293 cells expressing human Mac-1.

In some embodiments, antibodies and/or antigen binding fragments thereof, which inhibit interaction of leukocyte Mac-1 with platelet GP Ibα, can have a nanomolar binding affinity (K_{D}) to Mac-1 (human, rat, and/or mouse Mac-1) of less than about 10 nanomoles, less than about 5 nanomoles, or less than about 1 nanomoles and a nanomolar inhibitory constant (Ki) of leukocyte Mac-1 (human, rat, and/or mouse Mac-1) binding to platelet GP Ibα (human, rat, and/or mouse Mac-1) less than about 10 nanomoles, less than about 5 nanomoles, or less than about 1 nanomoles. Antibodies, which inhibit interaction of leukocyte Mac-1 with platelet GP Ibα, having a nanomolar binding affinity and nanomolar inhibitory binding constant can include rat monoclonal antibodies that are produced by clones 5C8, 10E2, 4F9, or 4B10.

Antibodies exhibiting nanomolar binding affinity to at least one of rat macrophage cell line (NR8383), mouse macrophage cell line (RAW) and/or 293 cells expressing human Mac-1 can be selected and analyzed for inhibition of thrombosis and/or vasculitis using known in vivo assays. For example, antibodies or antigen binding fragments thereof can be selected for their ability to inhibit or delay photochemical injury-induced arterial thrombosis in mice subjected to a Rose Bengal model of thrombosis. Antibodies or antigen binding fragments thereof can also be selected for their ability to inhibit or reduce thrombohemorrhagic vasculitis in a local Shwartzmann-like reaction (LSR) induced by successive lipopolysaccharide and cytokine injections into mouse skin.

The monoclonal antibodies can be chimeric, recombinant, humanized, or de-immunized. The antibody can also be an antibody fragment, such as a single chain antibody, IgG, F(ab)2, F(ab′)2, F(ab), F(ab′) fragment, or truncated antibody (e.g., Fc truncated antibody).

Antibodies or antigen binding fragments thereof, which inhibit interaction of leukocyte Mac-1 with platelet GP Ibα, can also be selected that bind to the same epitope on human α_{M}(P²⁰¹-K²¹⁷) as the anti-M2 antibodies described herein. Such antibodies can be identified based on their ability to cross-compete with anti-M2 antibodies in standard M2 binding assays. The ability of a test antibody to inhibit the binding of GPI bα to human Mac-1 demonstrates that the test antibody can compete with anti-M2 antibodies described for binding to M2 and thus binds to the same epitope on human M2.

In yet another aspect, an antibody of the invention comprises heavy and light chain variable regions comprising amino acid sequences that are homologous to the amino acid sequences of the preferred antibodies described herein, and wherein the antibodies retain the desired functional properties of the anti-M2 antibodies of the invention.

In certain aspects, an antibody of the invention can include a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein, or conservative modifications thereof, and wherein the antibodies retain the desired functional properties of the anti-M2 antibodies of the invention.

In some embodiments, the monoclonal antibody or antigen binding fragment can be a humanized monoclonal antibody or antigen binding fragment thereof that includes the 3 CDRs of the heavy chain variable domain and the 3 CDRs of light chain variable domain of a rat monoclonal antibody that specifically binds to SEQ ID NO: 1, SEQ ID NO: 2, or SEQ ID NO: 3. The rat monoclonal antibody can be produced by clones 5C8, 10E2, 4F9, or 4B10.

### Humanization and Display Technologies

The antibodies described can be used in a method of production and use of humanized anti-M2 antibodies, and antigen binding fragments thereof. Antibody humanization is a process that can generate engineered human antibodies with variable region (“V-region”) sequences that are substantially similar to actual human germ-line sequences, while retaining the binding specificity and affinity of a reference antibody, for example clones 5C8, 10E2, 4F9, or 4B10. This process can graft, for example, the CDR1, CDR2, and CDR3 regions of the heavy and the light chain sequences into humanized human framework that is both optimized and previously identified prior to the start of the grafting process. The variable region containing humanized framework can be produced into Fab, Fab′, or Fab2 single chain antigen-binding antibody fragments. The resulting engineered humanized antibody fragments can retain the binding specificity of the parent murine antibody for the antigen M2, and can have an equivalent or higher binding affinity for a specific antigen than the parent antibody. The engineered antigen binding fragments can have heavy and light chain V-regions with a high degree of amino acid sequence identity compared to the closest human germline antibody genes. For example, additional maturational changes can be introduced in the CDR3 regions of each chain during construction in order to identify antibodies with optimal binding kinetics.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art.

### Design and Generation of Other Therapeutics

Other therapeutic modalities beyond antibody moieties can be facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules. In connection with the generation of advanced antibody therapeutics, where complement fixation is a desirable attribute, it may be possible to sidestep the dependence on complement for cell killing using bispecifics, immunotoxins, or radiolabels, for example.

Bispecific antibodies can be generated that comprise (i) two antibodies one with a specificity to Mac-1 and another to a second molecule that are conjugated together, (ii) a single antibody that has one chain specific to M2 and a second chain specific to a second molecule, or (iii) a single chain antibody that has specificity to M2 and the other molecule. Such bispecific antibodies can be generated using techniques that are well known for example, in connection with (i) and (ii) (Fanger, M. W., R. F. Graziano, and P. M. Guyre, Production and use of anti-FcR bispecific antibodies. Immunomethods, 1994. 4(1): p. 72-81) and in connection with (iii) (Traunecker, A., A. Lanzavecchia, and K. Karjalainen, Janusin: new molecular design for bispecific reagents. Int J Cancer Suppl, 1992. 7: p. 51-2). In connection with the generation of therapeutic peptides, through the utilization of structural information related to Mac-1 and antibodies thereto, such as the antibodies of the invention (as discussed below in connection with small molecules) or screening of peptide libraries, therapeutic peptides can be generated that are directed against Mac-1.

### Therapeutic Uses

Antibodies or antigen binding fragments thereof, which inhibit interaction of leukocyte Mac-1 with platelet GP Ibα, can be used to treat diseases or disorders associated with aberrant leukocyte-platelet interactions in subjects, including humans, suffering from an acute or chronic pathological injury.

The diseases or disorders associated with aberrant leukocyte-platelet interactions can include:

Extracorporeal circulation diseases and disorders: Post-cardiopulmonary bypass inflammation, post-operative pulmonary dysfunction, cardiopulmonary bypass, hemodialysis, leukopheresis, plasmapheresis, plateletpheresis, heparin-induced extracorporeal LDL precipitation (HELP), postperfusion syndrome, extracorporeal membrane oxygenation (ECMO), cardiopulmonary bypass (CPB), post-perfusion syndrome, systemic inflammatory response, and multiple organ failure.

Cardiovascular diseases and disorders: acute coronary syndromes, Kawaski disease (arteritis), Takayasu's arteritis, Henoch-Schonlein purpura nephritis, vascular leakage syndrome, percutaneous coronary intervention (PCI), myocardial infarction, ischemia-reperfusion injury following acute myocardial infarction, atherosclerosis, vasculitis, immune complex vasculitis, vasculitis associated with rheumatoid arthritis (also called malignant rheumatoid arthritis), systemic lupus erythematosus-associated vasculitis, sepsis, arteritis, aneurysm, cardiomyopathy, dilated cardiomyopathy, cardiac surgery, peripheral vascular conditions, renovascular conditions, cardiovascular conditions, cerebrovascular conditions, mesenteric/enteric vascular conditions, diabetic angiopathy, venous gas embolus (VGE), Wegener's granulomatosis, heparin-induced extracorporeal membrane oxygenation, and Behcet's syndrome.

Bone/Musculoskeletal diseases and disorders: arthritis, inflammatory arthritis, non-inflammatory arthritis, rheumatoid arthritis, juvenile rheumatoid arthritis, systemic juvenile rheumatoid arthritis, osteoarthritis, osteoporosis, systemic lupus erythematosus (SLE), Behcet's syndrome, and Sjogren's syndrome.

Transplantation diseases and disorders: transplant rejection, xenograft rejection, graft versus host disease, xenotransplantation of organs or grafts, allotransplantation of organs or grafts, and hyperacute rejection.

Eye/Ocular diseases and disorders: wet and dry age-related macular degeneration (AMD), choroidal neurovascularization (CNV), retinal damage, diabetic retinopathy, diabetic retinal microangiopathy, histoplasmosis of the eye, uveitis, diabetic macular edema, diabetic retinopathy, diabetic retinal microangiopathy, pathological myopia, central retinal vein occlusion (CRVO), corneal neovascularization, retinal neovascularization, retinal pigment epithelium (RPE), histoplasmosis of the eye, and Purtscher's retinopathy.

Hemolytic/Blood diseases and disorders: sepsis, systemic inflammatory response syndrome” (SIRS), hemorrhagic shock, acute respiratory distress syndrome (ARDS), catastrophic anti-phospholipid syndrome (CAPS), cold agglutinin disease (CAD), autoimmune thrombotic thrombocytopenic purpura (TTP), endotoxemia, hemolytic uremic syndrome (HUS), atypical hemolytic uremic syndrome (aHUS), paroxysmal nocturnal hemoglobinuria (PNH), sepsis, septic shock, sickle cell anemia, hemolytic anemia, hypereosinophilic syndrome, and anti-phospholipid syndrome (APLS).

Respiratory/Pulmonary diseases and disorders: asthma, Wegener's granulomatosis, transfusion-related acute lung injury (TRALI), antiglomerular basement membrane disease (Goodpasture's disease), eosinophilic pneumonia, hypersensitivity pneumonia, allergic bronchitis bronchiecstasis, reactive airway disease syndrome, respiratory syncytial virus (RSV) infection, parainfluenza virus infection, rhinovirus infection, adenovirus infection, allergic bronchopulmonary aspergillosis (ABPA), tuberculosis, parasitic lung disease, adult respiratory distress syndrome, chronic obstructive pulmonary disease (COPD), sarcoidosis, emphysema, bronchitis, cystic fibrosis, interstitial lung disease, acute respiratory distress syndrome (ARDS), transfusion-related acute lung injury, ischemia/reperfusion acute lung injury, byssinosis, heparin-induced extracorporeal membrane oxygenation, anaphylactic shock, and asbestos-induced inflammation.

Central and Peripheral Nervous System/Neurological diseases and disorders: multiple sclerosis (MS), myasthenia gravis (MG), myasthenia gravis, multiple sclerosis, Guillain Bane syndrome, Miller-Fisher syndrome, stroke, reperfusion following stroke, Alzheimer's disease, multifocal motor neuropathy (MMN), demyelination, Huntington's disease, amyotrophic lateral sclerosis (ALS), Parkinson's disease, degenerative disc disease (DDD), meningitis, cranial nerve damage from meningitis, variant Creutzfeldt-Jakob Disease (vCJD), idiopathic polyneuropathy, brain/cerebral trauma (including, but not limited to, hemorrhage, inflammation, and edema), and neuropathic pain.

Trauma-induced injuries and disorders: hemorrhagic shock, hypovolemic shock, spinal cord injury, neuronal injury, cerebral trauma, cerebral ischemia reperfusion, crush injury, wound healing, severe burns, and frostbite.

Renal diseases and disorders: renal reperfusion injury, poststreptococcal glomerulonephritis (PSGN), Goodpasture's disease, membranous nephritis, Berger's Disease/IgA nephropathy, mesangioproliferative glomerulonephritis, membranous glomerulonephritis, membranoproliferative glomerulonephritis (mesangiocapillary glomerulonephritis), acute postinfectious glomerulonephritis, cryoglobulinemic glomerulonephritis, lupus nephritis, Henoch-Schonlein purpura nephritis, and renal cortical necrosis (RCN).

Reperfusion injuries and disorders of organs: including but not limited to heart, brain, kidney, and liver.

Reproduction and urogenital diseases and disorders: painful bladder diseases and disorders, sensory bladder diseases and disorders, spontaneous abortion, male and female diseases from infertility, diseases from pregnancy, fetomaternal tolerance, pre-eclampsia, urogenital inflammatory diseases, diseases and disorders from placental dysfunction, diseases and disorders from miscarriage, chronic abacterial cystitis, and interstitial cystitis.

Skin/Dermatologic diseases and disorders: burn injuries, psoriasis, atopic dermatitis (AD), eosinophilic spongiosis, urticaria, thermal injuries, pemphigoid, epidermolysis bullosa acquisita, autoimmune bullous dermatoses, bullous pemphigoid, scleroderma, angioedema, hereditary angioneurotic edema (HAE), erythema multiforme, herpes gestationis, Sjogren's syndrome, dermatomyositis, and dermatitis herpetiformis.

Gastrointestinal diseases and disorders: Crohn's disease, Celiac Disease/gluten-sensitive enteropathy, Whipple's disease, intestinal ischemia, inflammatory bowel disease, and ulcerative colitis.

Endocrine diseases and disorders: Hashimoto's thyroiditis, juvenile lymphocytic thyroiditis, stress anxiety, and other diseases affecting prolactin, growth or insulin-like growth factor, adrenocorticotropin release, pancreatitis, Addison's disease, diabetic conditions including, but not limited to, type 1 and type 2 diabetes, type I diabetes mellitus, sarcoidosis, diabetic retinal microangiopathy, non-obese diabetes (IDDM), angiopathy, neuropathy or retinopathy complications of IDDM or Type-2 diabetes, and insulin resistance.

Treatment of Malignancies: diseases and disorders arising from chemotherapeutics and radiation therapy.

In some embodiments, the diseases or disorders associated with aberrant leukocyte-platelet interactions can include post-cardiopulmonary bypass inflammation, pathogenic hypertrophy, cardiopulmonary bypass, percutaneous coronary intervention (PTCA), ischemia-reperfusion following acute myocardial infarction, myocardial infarction, atherosclerosis, heparin-induced extracorporeal membrane oxygenation LDL precipitation, extracorporeal membrane oxygenation, multiple organ failure, thrombosis formation, neointimal formation, neointimal hyperplasia, atherothrombosis, vasculitis, restinosis, stroke, angina, arthritis, and demyelinating disorders, such as multiple sclerosis and amyotrophic lateral sclerosis.

In one example, the Antibodies or antigen binding fragments thereof, which inhibit interaction of leukocyte Mac-1 with platelet GP Ibα, can be used in an extracorporeal circulation procedure, such as cardiopulmonary bypass (CPB) procedures on a subject. In these procedures, circulating blood can be passed from a blood vessel of the subject, through a conduit and back to a blood vessel of the subject. The conduit can have a luminal surface comprising a material capable of causing at least one of platelet activation, leukocyte activation, or platelet-leukocyte adhesion in the subject's blood. An antibody or antigen binding fragments thereof, which inhibit interaction of leukocyte Mac-1 with platelet GP Ibα, can be introduced into the subject's bloodstream in an amount effective to reduce platelet-leukocyte adhesion resulting from passage of the circulating blood through the conduit. The blood of the subject can be passed through the conduit before and/or during and/or after step introduction of the antibody or antigen binding fragments thereof.

### Therapeutic Administration and Formulations

It will be appreciated that the antibodies or antigen binding fragments thereof, which inhibit interaction of leukocyte Mac-1 with platelet GP Ibα, can be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as LIPFECTIN), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies described herein, if the active ingredient in the formulation is not inactivated by the formulation and the formulation is physiologically compatible and tolerable with the route of administration.

### Preparation of Antibodies

Antibodies or antigen binding fragments thereof, which inhibit interaction of leukocyte Mac-1 with platelet GP Ibα, can prepared in rats or mice using standard methods well know in the art. The monoclonal antibodies can be converted into a humanized version for therapeutic use. The antibodies can be made by contract or in house into humanized, chimeric, or recombinant for therapeutic use. The hybridoma cell lines discussed herein are readily generated by those of ordinary skill in the art, given the guidance provided herein. The antibodies produced by the subject cell lines do not generate an adverse response. Adverse response is defined as an unwanted response.

Antibodies or antigen binding fragments thereof, which inhibit interaction of leukocyte Mac-1 with platelet GP Ibα, can be expressed in cell lines other than hybridoma cell lines. Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive Mac-1 binding properties.

Human antibodies against a variety of antigens can also be produced from non-human transgenic mammals comprising human immunoglobulin loci. Typically these immunoglobulin loci can encode substantially human sequence antibodies, preferably 95% or more identical to human sequences, more preferably 98-99% or more identical, and most preferably 100% identical. The immunoglobulin loci can be rearranged or unrearranged, and can comprise deletions or insertions relative to the natural human immunoglobulin loci. The loci can include genetic elements (e.g., non-coding elements such as enhancers, promoters, and switch sequences, or coding elements such as mu constant region gene segments) from other species, and from non-immunoglobulin loci, that do not contribute substantially to the coding portion of secondary repertoire (non IgM) antibodies. The human immunoglobulin loci contained in these transgenic mammals preferably include unrearranged sequences of natural human heavy and human light chain loci. Usually, the endogenous immunoglobulin locus of such transgenic mammals is functionally inactivated (U.S. Pat. No. 5,589,369, Takeda, S. et al., 1993, EMBO J. 12:2329-2366; Jakobovits, A., et al., 1993, Proc. Natl. Acad. Sci. U.S.A. 90:2551-2555; Kitamura, D. and Rajewsky, K., 1992, Nature 356: 154-156; Gu, H. et al., 1991, Cell 65:47-54; Chen, J. et al., EMBO J. 12:821-830; Sun, W. et al., 1994, J. Immunol 152:695-704; Chen, J. et al., 1993, Intl. Immunology 5:647-656; Zou, X. et al., 1995, Eur. J. Immunol 25:2154-2162; Chen, J. et al., 1993 Intl. Immunology 5:647-656; Boudinot, P., et al, 1995, Eur. J. Immunol. 25:2499-2505; Chen, J. et al., 1993, Proc. Natl. Acad. Sci. 90:4528-4532; Roes, J. and Rajewsky, K., 1991, Intl. Immunology 3:1367-1371; Gu, H. et al., 1993, Cell 73:1155-1164; Taki, S. et al., 1993, Science 262: 1268-71; Kitamura, D. et al., 1991, Nature 350:423-6; Lutz, C. et al., 1998, Nature 393:797-801; Zou, Y. et al, 1994, Current Biology 4: 1099-1103; Chen, J. et al., 1993, EMBO J. 12:4635-4645; Serwe, M. and Sablitzky, F., 1993, EMBO J. 12:2321-2327; Sanchez, P. et al., 1994, Intl. Immunology 6:711-719; Zou, Y. et al., 1993, EMBO J. 12:811-820). Inactivation of endogenous immunoglobulin genes preferably can be achieved, e.g., by targeted homologous recombination. The exogenous human immunoglobulin loci can be associated the endogenous mouse chromosomes or can be of (e.g., part of, inserted within or attached to) an introduced transchromosome. Transchromosomes are introduced into a cell as a nonendogenous chromosome or chromosome fragment having a centromere and two telomeres. These transchromosomes commonly comprise telomere and centromere sequences and can comprise deletions relative to the parental intact chromosome. Transchromosomes can also comprise additional inserted sequences. A single transchromosome comprising two or three different immunoglobulin loci provides for genetic linkage of these loci which increases the fraction of transgenic offspring that are useful for making human antibodies. Preferred forms of transchromosomes are those described in detail in Tomizuka, K. et al., 2000, Proc. Natl. Acad. Sci. U.S.A. 97:722-727, Tomizuka, K. et al., 1997, Nature Genetics 16:133-143, and WO 97/07671, WO 98/37757 and WO 00/10383, each of which is incorporated by reference in its entirety for all purposes. Transchromosomes can also include integrated selectable markers and other sequences not found in the parent intact chromosome. In the event of recombination between a transchromosome and an endogenous mouse chromosome, sequences from the transchromosome are inserted or added to the endogenous mouse chromosome.

Transchromosomes can be modified by deletion, translocation, substitution and the like, as described in WO 98/37757, EP 0972445 and WO 00/10383, which are incorporated herein by reference for all purposes. For example, transchromosomes can be fragmented spontaneously in the course of introduction into mouse embryonic stem (ES) cells, fragmented by telomere-directed truncation and/or translocated by Cre/loxP site-specific recombination or similar methods. Such recombination or translocation events can be promoted by specifically inserting recombination sites (e.g., loxP sequences and others; see, e.g., Abuin, A. and Bradley, A., 1996, Mol. Cell Biol. 16: 1851-1856; Mitani, K. et al., 1995, Somat. Cell. Mol. Genet. 21:221-231; Li, Z. W. et al., 1996, Proc. Natl. Acad. Sci. U.S.A. 93:6158-6162; Smith, A J. et al., 1995, Nat. Genet. 9:376-385; Trinh, K R. and Morrison, S. L., 2000, J. Immunol. Methods 244:185-193; Sunaga, S. et al., 1997, Mol. Reprod Dev. 46: 109-113; Dymecki, S. M., 1996, Proc. Natl. Acad Sci. U.S.A. 93:6191-6196; Zou, Y R. et al., 1994, Curr. Biol. 4: 1099-1103; Rudolph, U. et al., 1993, Transgenic Res. 2:345-355; Rickert, R. C. et al., 1997, Nucleic Acids Res. 25:1317-1318). In the case of introduced loxP sites, expression of a transgene encoding the cre recombinase will promote recombination between the two loxP sites. Transchromosomes can also be a fusion chromosome consisting of different chromosome fragments as a result of the translocation described above. Transchromosomes can be autonomous. Autonomous transchromosomes are distinct from, are noncontiguous with, and are not inserted into the endogenous mouse chromosomes. These autonomous transchromosomes comprise telomere and centromere sequences that enable autonomous replication. Alternatively, transchromosome sequences can be translocated to mouse chromosomes after introduction into mouse cell nuclei. The endogenous mouse chromosomes include 19 autosomal chromosome pairs and the X and Y chromosomes.

Introduction of exogenous human immunoglobulin loci can be achieved by a variety of methods including, for example, microinjection of half-day embryo pronuclei, transfection of embryonic stem cells, or fusion of embryonic stem cells with yeast spheroplasts or micronuclei comprising transchromosomes. The transgenic mammals resulting from the processes described above are capable of functionally rearranging the introduced exogenous immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO 93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 48:1547-1553 (1994), Nature Biotechnology 14, 826 (1996), Kucherlapati, WO 91/10741 (1991), WO 94/02602 (1993), WO 96/34096 (1995), WO 96/33735 (1996), WO 98/24893 (1997), U.S. Pat. Nos. 5,939,598, 6,075,181, 6,114,598, Tomizuka, K. et al., 2000, Proc. Natl. Acad. Sci. U.S.A. 97:722-727, Tomizuka, K. et al., 1997, Nature Genetics 16:133-143, and Tomizuka, K., WO 97/07671, WO 98/37757, WO 00/10383, and JP 2000-42074 (each of which is incorporated by reference in its entirety for all purposes). Transgenic nonhuman mammals such as rodents are particularly suitable. Monoclonal antibodies can be prepared, e.g., by fusing B-cells from such mammals to suitable immortal cell lines using conventional Kohler-Milstein technology. Monoclonal antibodies can also be accessed directly from individual B cells, isolated from the medium, using PCR amplification of V regions (Schrader et al., 1997, U.S. Pat. No. 5,627,052). Alternatively, FACs sorted, or otherwise enriched B cell preparations can be used as a source of RNA or DNA for PCR amplification of V region sequences. Phage display methods (described below) can also be used to obtain human antibody sequences from immunized transgenic mice comprising human immunoglobulin loci. The human antibody V region sequences obtained by these methods can then be used to generate intact antibodies that retain the binding characteristics of the original parent antibodies. This process is described below.

A further approach for obtaining humanized antibodies is to screen a cDNA library from cells according to the general protocol outlined by Huse et al., 1989, Science 246:1275-1281. Such cells can be obtained from a human immunized with the desired antigen, fragments, longer polypeptides containing the antigen or fragments or anti-idiotypic antibodies. The cells can also be obtained from transgenic non-human animals expressing human immunoglobulin sequences. The transgenic non-human animals can be immunized with an antigen or collection of antigens. The animals can also be unimmunized. The V region encoding segments of the cDNA sequences are then cloned into a DNA vector that directs expression of the antibody V regions. Typically, the V region sequences are specifically amplified by PCR prior to cloning. Also typically, the V region sequences are cloned into a site within the DNA vector that is constructed so that the V region is expressed as a fusion protein. The collection of cloned V region sequences is then used to generate an expression library of antibody V regions. To generate an expression library, the DNA vector comprising the cloned V region sequences is used to transform eukaryotic or prokaryotic host cells. In addition to V regions, the vector can optionally encode all or part of a viral genome, and can comprise viral packaging sequences. In some cases, the vector does not comprise an entire virus genome, and the vector is then used together with a helper virus or helper virus DNA sequences. The expressed antibody V regions are found in, or on the surface of, transformed cells or virus particles from the transformed cells. This expression library, comprising the cells or virus particles, is then used to identify V region sequences that encode antibodies, or antibody fragments reactive with predetermined antigens. To identify these V region sequences, the expression library is screened or selected for reactivity of the expressed V regions with the predetermined antigens. The cells or virus particles comprising the cloned V region sequences, and having the expressed V regions, are screened or selected by a method that identifies or enriches for cells or virus particles that have V regions reactive (e.g., binding association or catalytic activity) with a predetermined antigen. For example, radioactive or fluorescent labeled antigen that then binds to expressed V regions can be detected and used to identify or sort cells or virus particles. Antigen bound to a solid matrix or bead can also be used to select cells or virus particles having reactive V regions on the surface. The V region sequences thus identified from the expression library can then be used to direct expression, in a transformed host cell, of an antibody or fragment thereof, having reactivity with the predetermined antigen. The protocol described by Huse is rendered more efficient in combination with phage-display technology. See, e.g., Dower et al., WO 91/17271 and McCafferty et al., WO 92/01047, U.S. Pat. Nos. 5,871,907, 5,858,657, 5,837,242, 5,733,743, and 5,565,332, (each of which is incorporated by reference in its entirety for all purposes). In these methods, libraries of phage are produced in which members (display packages) display different antibodies on their outer surfaces. Antibodies are usually displayed as Fv or Fab fragments. Phage displaying antibodies with a desired specificity can be selected by affinity enrichment to the antigen or fragment thereof. Phage display combined with immunized transgenic non-human animals expressing human immunoglobulin genes can be used to obtain antigen specific antibodies even when the immune response to the antigen is weak.

In a variation of the phage-display method, human antibodies having the binding specificity of a selected rat or murine antibody can be produced. See, for example, Winter, WO 92/20791. In this method, either the heavy or light chain variable region of the selected rat or murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for the selected are selected. Artificial antibodies that are similar to human antibodies can be obtained from phage display libraries that incorporate random or synthetic sequences, for example, in CDR regions.

The heavy and light chain variable regions of chimeric, humanized, or human antibodies can be linked to at least a portion of a human constant region by various well-known methods (see, e.g., Queen et al., 1989, Proc. Natl. Acad. Sci. U.S.A. 86:10029-10033 and WO 90/07861; these references and references cited therein are herein incorporated by reference for all purposes). The choice of constant region depends, in part, whether antibody-dependent complement and/or cellular mediated toxicity is desired. For example, isotypes IgG₁ and IgG₃ usually have greater complement binding activity than isotypes IgG₂ or IgG₄. Choice of isotype can also affect passage of antibody into the brain. Light chain constant regions can be lambda or kappa. Antibodies can be expressed as tetramers containing two light and two heavy chains, as separate heavy chains, light chains, as Fab, Fab′, F(ab′)2, and Fv, or as single chain antibodies in which heavy and light chain variable domains are linked through a spacer.

For some applications, non IgG antibodies can be useful. For example, where multivalent antibody complexes are desired, IgM and IgA antibodies can be used.

Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, FROM GENES TO CLONES, (VCH Publishers, NY, 1987). A number of suitable host cell lines capable of secreting intact heterologous proteins have been developed in the art, and include CHO cell lines, various COS cell lines, HeLa cells, L cells and myeloma cell lines. Preferably, the cells are nonhuman. Expression vectors for these cells can include expression control sequences, such as an origin of replication, a promoter, an enhancer (Queen et al., 1986, Immunol. Rev. 89:49), and necessary processing information sites, such as ribosome binding sites, RNA splice sites, polyadenylation sites, and transcriptional terminator sequences.

Alternatively, antibody coding sequences can be incorporated in transgenes for introduction into the genome of a transgenic animal and subsequent expression in the milk of the transgenic animal (see, e.g., U.S. Pat. Nos. 5,741,957, 5,304,489, 5,849,992). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, Protein Purification (Springer-Verlag, NY, 1982)).

One example of a method of preparing a recombinant polyclonal antibody is by making polyclonal antibody libraries (PCAL), for instance as disclosed in U.S. Pat. No. 5,789,208 (to J. Sharon) which is hereby incorporated by reference in its entirety.

More specifically, the polyclonal antibody included in the pharmaceutical composition may be prepared by immunizing an animal, preferably a mammal, with an antigen of choice followed by the isolation of antibody-producing B-lymphocytes from blood, bone marrow, lymph nodes, or spleen. Alternatively, antibody-producing cells may be isolated from an animal and exposed to an antigen in vitro against which antibodies are to be raised. The antibody-producing cells may then be cultured to obtain a population of antibody-producing cells, optionally after fusion to an immortalized cell line such as a myeloma.

A combinatorial library may be prepared from immunized B lymphocytes by associating V_{L} and V_{H} randomly in a cloning vector. Thus, the recombinant polyclonal antibody is generated under such conditions that the immunoglobulin heavy chain variable region and light chain variable region gene segments are linked together randomly in order to allow for the bulk transfer of variable region light chain and heavy chain gene pairs from one vector to another, while allowing stable pairing of specific immunoglobulin variable region light chain and heavy chain gene segments as they are present upon selection from a parental library of immunoglobulin variable region light chain and heavy chain gene segment pairs encoding antibody molecules capable of reacting with or binding to an allergen.

Single cell PCR may be used in an attempt to retain the native pairing of V_{L} and V_{H} in the single cell. In this case antibody-producing B-lymphocytes which have been isolated from animals or humans may be fixed with a fixative solution or a solution containing a chemical such as formaldehyde, glutaraldehyde or the like. The cells are then permeabilized with a permeabilization solution comprising for example a detergent such as Brij, Tween, polysorbate, Triton X-100, or the like. The fixing and permeabilization process should provide sufficient porosity to allow entrance of enzymes, nucleotides and other reagents into the cells without undue destruction of cellular compartments or nucleic acids therein. Addition of enzymes and nucleotides may then enter the cells to reverse transcribe cellular V_{H} and V_{L} mRNA into the corresponding cDNA sequences.

Upon reverse transcription, the resulting cDNA sequences may be amplified by PCR using primers specific for immunoglobulin genes and, in particular, for the terminal regions of the V_{H} and V_{L} nucleic acids. PCR procedures may be followed as disclosed in, e.g., U.S. Pat. No. 4,683,195. Preferably, the cDNAs are PCR amplified and linked in the same reaction, using, in addition to the cDNA primers, one primer for the 5′ end of the V_{H} region gene and another for the 5′ end of the V_{L} gene. These primers also contain complementary tails of extra sequence, to allow the self-assembly of the V_{H} and V_{L} genes. After PCR amplification and linking, the chance of getting mixed products, in other words, mixed variable regions, is minimal because the amplification and linking reactions were performed within each cell. The amplified sequences are linked by hybridization of complementary terminal sequences. After linking, sequences may be recovered from cells. For example, after linking, cells can be washed in a solution of sodium dodecyl sulfate (SDS). The SDS precipitates out of the cells after incubation on ice and the supernatant can be electrophoresed into an agarose or acrylamide gel. Alternatively, or in combination with the SDS process, using a reagent such as digoxigenin-linked nucleotides, DNA products synthesized will remain within the cell and be amplified. The linked product is recovered upon electrophoresis of the supernatant.

After electrophoresis of the supernatant, the gel slice corresponding to the appropriate molecular weight of the linked product is removed and the DNA isolated on, for example, silica beads. The recovered DNA can be PCR amplified using terminal primers, if necessary, and cloned into vectors which may be plasmids, phages, cosmids, phagamids, viral vectors or combinations thereof. Convenient restriction enzyme sites may be incorporated into the hybridized sequences to facilitate cloning. These vectors may also be saved as a library of linked variable regions for later use.

The linked V_{H} and V_{L} region genes may be PCR amplified a second time using terminal nested primers, yielding a population of DNA fragments, which encode the linked V_{H} and V_{L} genetic regions. The grouping of V_{H} and V_{L} combinations is an advantage of this process and allows for the in mass or batch transfer of all clones and all DNA fragments during this and all cloning procedures.

The recombinant polyclonal antibody may be generated under such conditions that the immunoglobulin heavy chain variable region and light chain variable region gene segments are linked together in a head-to head orientation, in order to allow for the bulk transfer of variable region light chain and heavy chain pairs from one vector to another, including from phage to vector, and including from the cell of origin to phage or vector, resulting in a stable pairing of specific immunoglobulin variable region light chain and heavy chains gene segments as they are found in the original polyclonal immune response of the animal or human individual.

It may sometimes be desirable to treat the variable region gene sequences with a mutating agent. Mutating agents create point mutations, gaps, deletions or additions in the genetic sequence which may be general or specific, or random or site directed. Useful mutating agents include ultraviolet light, gamma irradiation, chemicals such as ethidium bromide, psoralen and nucleic acid analogs, or DNA modifying enzymes such as restriction enzymes, transferases, ligases and specific and nonspecific nucleases and polymerases. Moreover, it may be feasible to use mutator strains. In particular, random mutations may be introduced in the CDRs of the V_{H} and V_{L} region genes by oligonucleotide directed mutagenesis. Mutations introduced into the gene sequence will ultimately increase library complexity and diversity as well as affinity for antigen which may further increase the library's usefulness in treatment. Furthermore, such mutagenesis may be used on a single V_{H} and V_{L} pair or on a defined group of such pairs to generate a library de novo.

Vectors are transformed into suitable host cells and the cultures amplified to expand the different populations of vectors that comprise the library. Host cells for prokaryotic vectors may be a culture of bacteria such as Escherichia coli. Host cells for eukaryotic vectors may be a culture of eukaryotic cells such as any mammalian, insect or yeast cell lines adapted to tissue culture. Bacterial cells are transformed with vectors by calcium chloride-heat shock or electroporation, although many other transformation procedures would also be acceptable. Eukaryotic cells are transfected with calcium phosphate precipitation or electroporation, although many other transformation procedures would also be acceptable. The DNA fragments may be cloned into prokaryotic or eukaryotic expression vectors, chimeric vectors or dual vectors. The expression vector may be a plasmid, cosmid, phage, viral vector, phagemid and combinations thereof, but is preferably a phage display vector wherein the recombinant product is expressed on the phage surface to facilitate screening and selection. Useful transcriptional and translational sites may be placed on the expression vector including RNA polymerase recognition regions such as a TATA box site, a CAT site, an enhancer, appropriate splicing sites, if necessary, a AT rich terminal region and a transcription initiation site. Useful sites to facilitate translation include translational start and stop sites and ribosome binding sites. Typically, some of the more useful sites for efficient eukaryotic expression, such as the SV40, CMV, HSV or baculovirus promoter/enhancer region, are derived from viruses. The resulting recombinant antibody may be of the murine class IgG₁, IgG₂ₐ, IgG_{2b}, IgM, IgA, IgD or IgE, the human classes IgG₁, IgG₂, IgG₃, IgG₄, IgM, IgA₁, IgA₂, IgD or IgE, or combinations or fragments thereof. Preferably, the chimeric antibody library is composed of primarily IgG antibodies or Fab antibody fragments.

### Treatment Methods

The antibodies or antigen binding fragments thereof, which inhibit interaction of leukocyte Mac-1 with platelet GP Ibα, can be administered to an individual in a formulation with a pharmaceutically acceptable excipient(s). A wide variety of pharmaceutically acceptable excipients are known in the art and need not be discussed in detail herein.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, a subject antibody may be administered to the host using any convenient means capable of resulting in the desired therapeutic effect. Thus, the antibody can be incorporated into a variety of formulations for therapeutic administration. More particularly, a subject antibody can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of a subject antibody can be achieved in various ways, including oral, buccal, rectal, parenteral, intraperitoneal, intradermal, subcutaneous, intramuscular, transdermal, intranasal, pulmonary, intratracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

A subject antibody can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of a subject antibody calculated in an amount sufficient to produce the desired effect in association with a pharmaceutically acceptable diluent, carrier or vehicle.

A subject antibody is administered to an individual at a frequency and for a period of time so as to achieve the desired therapeutic effect. For example, a subject antibody is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), or substantially continuously, or continuously, over a period of time ranging from about one day to about one week, from about two weeks to about four weeks, from about one month to about two months, from about two months to about four months, from about four months to about six months, or longer.

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### Example

Disrupting the binding of Mac-1 to GP Ibα prevents MI and prolongs survival in Dahl salt-sensitive rats transgenic for human cholesteryl ester transfer protein. Given the accumulating evidence supporting a role for neutrophils in plaque rupture, thrombosis, and acute coronary syndrome, we proposed a pivotal role for Mac-1 in this biology and hypothesized that disrupting Mac-1 binding to GP Ibα would prevent MI and improve survival in Dahl salt-sensitive rats transgenic for human cholesteryl ester transfer protein (designated Tg(hCETP)^{DS} rats). Male Tg(hCETP)^{DS} rats fed regular rat chow show age-dependent severe combined hyperlipidemia, coronary artery atherosclerotic lesions, and MI at 9-12 months, and subsequent decreased survival compared with that of control non-trangenic Dahl S rats.

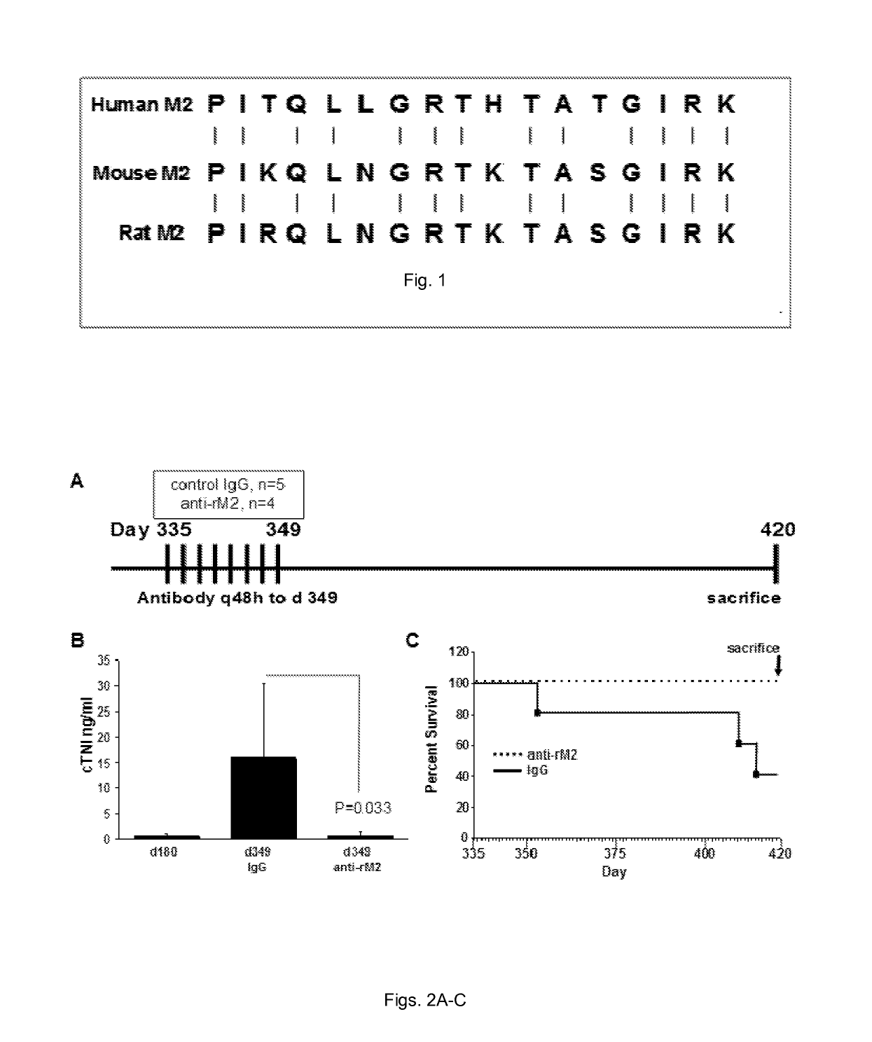

As a first step in targeting rat Mac-1-GP Ibα for in vivo experiments, we generated MAbs to the rat M2 site by immunizing mice with KLH coupled to (C)PIRQLNGRTKTASGIRK, (SEQ ID NO: 3) the rat M2 peptide sequence corresponding to human α_{M}(P²⁰¹-K²¹⁷). This rat sequence is highly homologous to the mouse (16 out of 17 residues identical) and human (13 out of 17 residues identical) α_{M}(P²⁰¹-K²¹⁷) sequences (FIG. 1). MAbs were screened for their ability to bind to rat M2-CGGG coated to high binding ELISA plate. MAbs were also screened for their ability to bind to rat macrophage cell line (NR8383), mouse macrophage cell line (RAW), and 293 cells expressing human Mac-1. MAbs were also tested for their ability to block leukocyte adhesion to platelets using whole blood from the particular species of interest. We obtained a MAb (clone 5C8), designated anti-rM2, that inhibited rat leukocyte arrest on adherent rat platelets under flow (maximal % inhibition=91±1, P<0.01). Clone 4F9, designated anti-mM2, inhibited mouse leukocyte arrest on mouse platelets (maximal % inhibition=76±1, P<0.01). Male Tg(hCETP)^{DS} rats were treated with anti-rM2 MAb (n=4) or mouse IgG control (n=5) (1 mg/kg IP q48 h×10 doses) beginning at day 335 of age and survival assessed over time to construct Kaplan-Meier curves comparing the 2 treatment groups. During antibody treatment, anticoagulated blood samples were obtained to monitor cardiac troponin (cTNI) release as an indicator of myonecrosis. As shown in FIG. 2, treatment with anti-rM2 prevented MI as assessed at day 349 (cTNI: 0.65±0.53 compared to 15.1±14.5 ng/ml, P=0.033) and prolonged survival (0 out of 4 dead treated with anti-rM2 compared to 3 out of 5 dead treated with IgG alone; P=0.07) at day 425 of planned sacrifice. These show that Mac-1 plays a critical role in atherothrombosis.

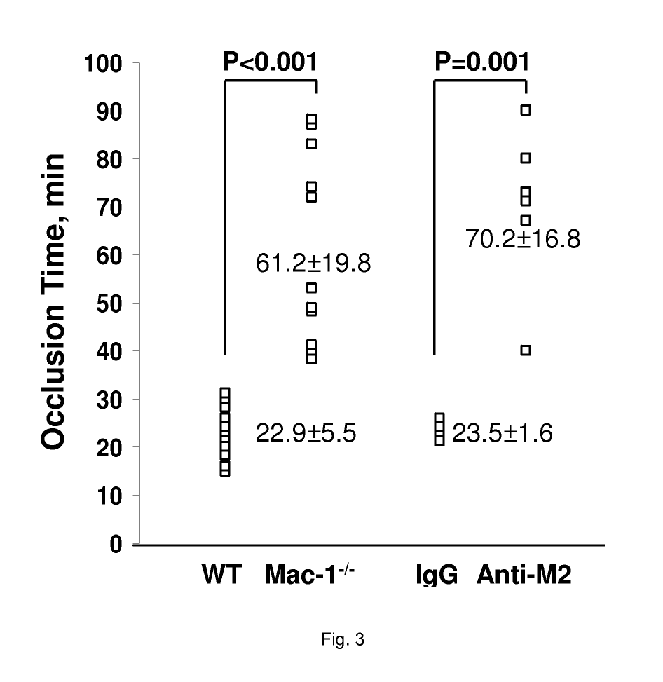

Having demonstrated that GP Ibα engagement of Mac-1 initiates pro-thrombotic and pro-inflammatory signals in platelets leading to phosphorylation of Akt and activation of αIIβ₃, we pursued studies investigating the role of Mac-1-GP Ibα in thrombus formation in vivo using a photochemical carotid artery thrombosis model (FIG. 3). The right common carotid artery of WT and Mac-1^{-/-} mice was isolated, and a vascular flow probe (Transonic Systems) was applied to monitor blood flow. Rose Bengal at a concentration of 50 mg/mL in phosphate-buffered saline was injected into the tail vein to administer a dose of 50 mg/kg. The mid portion of the common carotid artery was then illuminated with a 1.5-mW green light laser (540 nm; Melles Griot Inc) until an occlusive thrombus was formed. The time required to form an occlusive thrombus, defined as absence of blood flow for 3 minutes or more, was recorded. Mean time to occlusion was prolonged significantly in Mac-1^{-/-} (61.2±19.8 min) compared to WT (22.9±5.5 min, P<0.001) mice and in anti-rM2 (70.2±16.8 min) compared to control IgG (23.5±1.6 min, P=0.001) treated WT mice, indicating that Mac-1, and in particular the interaction between leukocyte Mac-1 and platelet GP Ibα regulates arterial thrombosis.

### Clones

5C8: Isotype: IgG1/k. High binding signal to rat M2 sequence. Weaker binding to rat macrophages, mouse macrophages and human 293 human Mac-1 expressing cells. 5C8 inhibited adhesion of rat PMN to rat platelets in static binding assay. Positive staining of rat leukocytes of rat tissue by immunohistochemistry.

10E3: Isotype: IgG1/k. High binding signal to rat M2 sequence. Weaker binding to rat macrophages, mouse macrophages and human 293 human Mac-1 expressing cells.

4F9: Isotype: IgG2a. Binds to mouse macrophages. Also recognizes rat M2 sequence. 4F9 inhibited mouse leukocyte arrest on adherent mouse platelets under flow conditions.

4B10: Isotype:IgM/k. ELISA: It binds strongly to 293 cells expressing human Mac-1. In addition, it binds to rat M2 sequence, rat macrophages, and mouse macrophage. 4B10 inhibits human leukocyte rolling over adherent human platelets in anticoagulated human whole blood under flow.

What has been described above includes examples and implementations of the present invention. Because it is not possible to describe every conceivable combination of components, circuitry or methodologies for purposes of describing the present invention, one of ordinary skill in the art will recognize that many further combinations and permutations of the present invention are possible. Accordingly, the present invention is intended to embrace all such alterations, modifications and variations that fall within the spirit and scope of the appended claims.

Having described the invention, the following is claimed:

1. A method of treating a subject having or at risk of a disease or disorder associated with aberrant leukocyte-platelet interactions, the method comprising:
administering to the subject a therapeutically effective amount of a monoclonal antibody or antigen binding portion thereof that specifically binds to at least one of SEQ ID NO: 1, SEQ ID NO: 2, or SEQ ID NO: 3 to inhibit leukocyte arrest on adherent platelets in the subject.

2. The method of claim 1, wherein the antibody or antigen binding fragment thereof has a nanomolar binding affinity (K_{D}) to Mac-1 of less than about 10 nanomoles.

3. The method of claim 1, wherein the antibody or antigen binding fragment thereof has a nanomolar inhibitory constant (Ki) of leukocyte Mac-1 binding to platelet GP Ibα less than about 10 nanomoles.

4. The method of claim 1, wherein the antibody or antigen binding fragment thereof includes the 3 CDRs of the heavy chain variable domain and the 3 CDRs of light chain variable domain of a rat monoclonal antibody that specifically binds to SEQ ID NO: 1, SEQ ID NO: 2, or SEQ ID NO: 3.

5. The method of claim 4, wherein the rat monoclonal antibody is produced by at least one of clones 5C8, 10E2, 4F9, or 4B10.

6. The method of claim 1, wherein the diseases or disorders associated with aberrant leukocyte-platelet interactions are selected from the group consisting of post-cardiopulmonary bypass inflammation, pathogenic hypertrophy, cardiopulmonary bypass, percutaneous coronary intervention (PTCA), ischemia-reperfusion following acute myocardial infarction, myocardial infarction, atherosclerosis, heparin-induced extracorporeal membrane oxygenation LDL precipitation, extracorporeal membrane oxygenation, multiple organ failure, thrombosis formation, neointimal formation, neointimal hyperplasia, atherothrombosis, vasculitis, restinosis, stroke, angina, arthritis, and demyelinating disorders.

7. The method of claim 1, wherein the diseases or disorders associated with aberrant leukocyte-platelet interactions is thrombosis and/or myocardial infarction.

8. A method of treating a subject having or at risk of myocardial infarction, the method comprising:
administering to the subject a therapeutically effective amount of a monoclonal antibody or antigen binding portion thereof that specifically binds to at least one of SEQ ID NO: 1, SEQ ID NO: 2, or SEQ ID NO: 3 to inhibit leukocyte arrest on adherent platelets in the subject.

9. The method of claim 8, wherein the antibody or antigen binding fragment thereof has a nanomolar binding affinity (K_{D}) to Mac-1 of less than about 10 nanomoles.

10. The method of claim 8, wherein the antibody or antigen binding fragment thereof has a nanomolar inhibitory constant (Ki) of leukocyte Mac-1 binding to platelet GP Ibα less than about 10 nanomoles.

11. The method of claim 8, wherein the antibody or antigen binding fragment thereof includes the 3 CDRs of the heavy chain variable domain and the 3 CDRs of light chain variable domain of a rat monoclonal antibody that specifically binds to SEQ ID NO: 1, SEQ ID NO: 2, or SEQ ID NO: 3.

12. The method of claim 11, wherein the rat monoclonal antibody is produced by at least one of clones 5C8, 10E2, 4F9, or 4B10.

13. A method of treating a subject having or at risk of a disease or disorder associated with aberrant leukocyte-platelet interactions, the method comprising:
administering to the subject a therapeutically effective amount of a monoclonal antibody or antigen binding portion thereof that specifically binds to at least one of SEQ ID NO: 1, SEQ ID NO: 2, or SEQ ID NO: 3 to inhibit leukocyte arrest on adherent platelets in the subject, wherein the antibody or antigen binding fragment thereof includes the 3 CDRs of the heavy chain variable domain and the 3 CDRs of the light chain variable domain of an antibody produced by at least one of clones 5C8, 10E2, 4F9, or 4B10.

14. The method of claim 13, wherein the antibody or antigen binding fragment thereof has a nanomolar binding affinity (K_{D}) to Mac-1 of less than about 10 nanomoles.

15. The method of claim 13, wherein the antibody or antigen binding fragment thereof has a nanomolar inhibitory constant (Ki) of leukocyte Mac-1 binding to platelet GP Ibα less than about 10 nanomoles.

16. The method of claim 13, wherein the diseases or disorders associated with aberrant leukocyte-platelet interactions are selected from the group consisting of post-cardiopulmonary bypass inflammation, pathogenic hypertrophy, cardiopulmonary bypass, percutaneous coronary intervention (PTCA), ischemia-reperfusion following acute myocardial infarction, myocardial infarction, atherosclerosis, heparin-induced extracorporeal membrane oxygenation LDL precipitation, extracorporeal membrane oxygenation, multiple organ failure, thrombosis formation, neointimal formation, neointimal hyperplasia, atherothrombosis, vasculitis, restinosis, stroke, angina, arthritis, and demyelinating disorders.

17. The method of claim 13, wherein the diseases or disorders associated with aberrant leukocyte-platelet interactions is thrombosis and/or myocardial infarction.


SEQUENCE LISTING (3 sequences)

SEQ ID NO: 1 - protein, 17 aa, Homo sapiens
PITQLLGRTHTATGIRK

SEQ ID NO: 2 - protein, 17 aa, Mus musculus
PIKQLNGRTKTASGIRK

SEQ ID NO: 3 - protein, 17 aa, Rattus rattus
PIRQLNGRTKTASGIRK